(19) United States
(12) Patent Application Publication
(10) Pub. No.: US 2024/0400611 A1
(43) Pub. Date: Dec. 5, 2024

(54) A PROCESS OF FLOCCULATION TO PURIFY CRUDE FERMENTATION BROTH

(71) Applicant: BIOCON BIOLOGICS LIMITED, Karnataka (IN)

(72) Inventors: Azimoddin Minyasab Shaikh, Bengaluru (IN); Partha Pratim Hazra, Bengaluru (IN); Vibhava Shukla, Bengaluru (IN); Kartik Shivappa Ganiger, Bengaluru (IN)

(73) Assignee: BIOCON BIOLOGICS LIMITED, Bengaluru (IN)

(21) Appl. No.: 18/696,764

(22) PCT Filed: Sep. 15, 2022
(86) PCT No.: PCT/IN2022/050827

§ 371 Date: Mar. 28, 2024

(30) Foreign Application Priority Data
Oct. 1, 2021 (IN) 202141044738

(51) Int. Cl. C07K 1/18; C07K 1/14; C07K 14/62; C12P 21/02; C12R 1/84

(52) U.S. Cl. CPC C07K 1/18; C07K 1/145; C07K 14/62; C12P 21/02; C12R 2001/84

(57) ABSTRACT

The present invention relates to process of flocculation to purify recombinant proteins from a crude fermentation broth of Insulin and Insulin analogues or derivatives at a manufacturing scale in the presence of urea and Triton-X-100 at specific pH. The flocculation process is followed by at least one centrifugation and pH adjustment step for further clarification of the broth. This is further followed by filtration for final removal of any floccules present and chromatography steps to capture the pure protein. A high product recovery of more than 95% was achieved using this process.

### FIELD OF INVENTION

A process as disclosed herein falls in the field of downstream processing. The process relates to purification of proteins from a fermentation broth. More particularly, provided is a process of flocculation for purification of protein from protein suspensions containing soluble and insoluble components.

### BACKGROUND

The following discussion of the background is merely provided to aid the reader in understanding the invention and is not admitted to describe or constitute prior art to the present invention.

Production of recombinant proteins using Pichia pastoris as a host cell has been a wide industrial practice. Therapeutic recombinant proteins like insulin, its analogues and derivatives are peptides with the size of around 5-7 kDa. These peptides have been expressed in Pichia pastoris in the form of precursor molecules and are secreted externally in the fermentation supernatants. Post fermentation harvest, cells are separated by means of continuous centrifugation. Post centrifugation the supernatant contains significant amount of Pichia cells, cell debris, Pichia related pigments and other media related impurities. This crude cannot be directly loaded onto a chromatographic column. The insoluble solids as well as many soluble impurities cause significant damage to purification columns and can severely interfere with the purification process resulting in lower column life and compromised purification performance, if being carried forward through capture and all the way up to the purification steps.

A physical separation method such as membrane filtration was first used for clarifying a fermentation supernatant. Microfiltration is another widely used technique for clarification of feeds with high solid content. Extensive development was done to develop clarification process using microfiltration. A process was developed using 0.1-micron Micro-filtration membrane. Post microfiltration, filtrate was further concentrated through ultrafiltration to overcome the dilution encountered during microfiltration. This process was scaled-up with a total area of 100 m² to filter supernatant volume of 20-22KL. Ionic polymers have also been used to modify fermentation media to enhance the removal of impurities from process streams in applications such as depth filtration or membrane absorber.

As mentioned in Europe patent no. 1934242, conventional biopharmaceutical protein purification methods used to remove cells and cellular debris are not always effective and sometimes significantly bind the product of interest, increasing the overall process time which can be challenging during the scaling up of the operational procedure. Any improvements that allow for quicker recovery times and/or greater recovery is desirable as it reduces costs associated with manufacturing protein therapeutics.

Therefore, there is still a need for an efficient purification process.

### SUMMARY OF THE INVENTION

Provided is a process of purifying a recombinant protein in a fermentation broth. The process involves flocculation of impurities.

The disclosed process is a downstream protein recovery process by providing a flocculation step after the harvest of the culture broth. In some embodiments the process is a process for purifying a protein of interest from a yeast fermentation broth comprising a) flocculating the fermentation supernatant; b) performing at least one separation step.

Provided is a process of purifying a recombinant protein in a fermentation broth. The process includes adding urea and a non-ionic detergent to the fermentation broth. The process further includes adjusting the pH of the fermentation broth to a value in the range from pH 2 to 4.5 or to a value in the range from pH 7.5 to 8.5. In some embodiments urea and the non-ionic detergent are added prior to adjusting the pH value. In some embodiments the pH value is adjusted prior to adding urea and the non-ionic detergent. The process also includes incubating the fermentation broth for 30 minutes or more. The process further includes separating insoluble matter from the fermentation broth. By separating the insoluble matter, a supernatant is obtained.

In some embodiments, the process furthermore includes adding urea and a non-ionic detergent to the supernatant. In such embodiments the process also includes adjusting the pH of the supernatant to a value in the range from pH 2 to 4.5 or to a value in the range from pH 7.5 to 8.5. In some embodiments urea and the non-ionic detergent are added prior to adjusting the pH value. In some embodiments the pH value is adjusted prior to adding urea and the non-ionic detergent. Such embodiments of the process furthermore include incubating the supernatant for at least 30 minutes. The process in such embodiments also includes separating insoluble matter from the supernatant.

The urea is in some embodiments added to a final concentration of 0.1-0.3M. In some embodiments, urea is added to a final concentration of 0.15 to 0.25M.

In some embodiments the non-ionic detergent is added to a concentration in the range of up to 1% (v/v).

Separating insoluble matter from the fermentation broth and/or the supernatant is in some embodiments performed by centrifugation or filtration. Separating insoluble matter from the fermentation broth and/or the supernatant comprises exposing the broth to depth filtration.

In some embodiments the non-ionic detergent is based on polyoxyethylene as a polar portion and contains an alkylphenyl moiety as a non-polar portion. In some embodiments the non-ionic detergent is based on a fatty acid ester with a polyoxyethylene chain having terminal hydroxy groups as a polar portion, with the alkyl chain of the fatty acid defining a non-polar portion. In some embodiments the non-ionic detergent is based on a maltoside or a glucoside as a polar portion and contains an alkyl chain as a non-polar portion. In some embodiments the non-ionic detergent is selected from the group of Triton, Tween or Brij series.

In some embodiments the non-ionic detergent is Triton X-100, (IUPAC name 2-[4-(2,4,4-trimethylpentan-2-yl)phenoxy]ethanol). In some embodiments 2-[4-(2,4,4-trimethylpentan-2-yl)phenoxy]ethanol is added to a final concentration of 0.1-0.4%, such as 0.15%.

In some embodiments the recombinant protein is insulin or an insulin analogue or derivative. The insulin analogue may for instance be Insulin Glargine, Insulin Lispro. Insulin Aspart or oral insulin tregopil.

In some embodiments the recombinant protein has been produced by a yeast. The yeast may for example be Pichia pastoris.

Adjusting the pH of the fermentation broth or of the supernatant is performed by adding a suitable base such as sodium hydroxide or potassium hydroxide. Sodium hydroxide or potassium hydroxide may for example be added in the form of a solution that is 2.5M.

In some embodiments the process is part of a purification process that includes cation exchange chromatography as the final capture step. In some embodiments of such a purification process more than 99% of urea and the non-ionic detergent are removed after the cation exchange chromatography. In some embodiments of such a purification process more than 95% of the produced protein is recovered.

In some embodiments the process is a process of flocculation for purification of recombinant protein from a crude fermentation broth comprising steps of:
a. Production of recombinant protein using Pichia pastoris as a suitable host;
b. Flocculating the impurities in the fermentation broth at pH of range of 2 to 4.5 and/or pH range of 7.5 to 8.5 by addition of Urea and Triton X-100;
c. Removal of the floccules by centrifugation or filtration;
d. Readjusting the pH to be in the range of 2 to 2.5;
e. Final capture of the protein by chromatography.

The process in some embodiments relates to purification of recombinant protein such as insulin or insulin analogues such as insulin Glargine, Insulin Lispro and Insulin Aspart

In some embodiments of the process of flocculation, the floccules are removed by centrifugation and depth filtration, and final capture of protein is achieved by Cation exchange chromatography.

### BRIEF DESCRIPTION OF THE DRAWINGS

FIG. 1 represents a flow chart of an illustrative process of flocculation that was applied in the primary treatment of Glargine supernatant.

FIG. 2A depicts the NTU stability at Room Temperature hold for Glargine FIG. 2B depicts the NTU stability at cold temperature hold for Glargine FIG. 3 represents a flow chart of an illustrative process of flocculation that was applied in the primary treatment of Lispro supernatant.

FIG. 4A depicts the NTU stability data for Lispro at RT hold, pH 2.0±0.2

FIG. 4B depicts the NTU stability data for Lispro at cold temperature hold, pH 2.0±0.2

FIG. 5A depicts the NTU stability data for Lispro at RT hold, pH 4±0.2

FIG. 5B depicts the NTU stability data for Lispro at cold temperature hold, pH 4±0.2

### DETAILED DESCRIPTION

Where a recombinant protein is secreted into the cell culture medium, downstream processing of the recombinant protein begins with harvesting the respective medium and separating it from cells that were expressing the protein. This recovery step includes the removal of cell debris, as well as the removal of any micro-particulates and colloidal material. Thereafter, bulk contaminants, mainly proteins, can be removed, and finally a polishing step, removing trace contaminants, can be applied. The process provided herein relates to a purification step that follows the initial removal of cells that had expressed the protein.

The process disclosed herein is a result of efforts to counter the problems faced in the methods that exist in prior art. The impurities present in the supernatant were screened for a broad flocculation pH range. During such a study, it was observed that these impurities have a tendency to flocculate at a pH range of 2 to 4.5 and 7.5 to 8.5. Flocculation majorly happened due to alteration in pH levels and was partly assisted by lyotropic salts, which were formed in situ. Attempts were made to accelerate the flocculation or increase the extent of flocculation by means of external flocculation agents like calcium chloride, however it was observed that pH-based flocculation was sufficient and caused significant change in distribution of the particulate matter present in supernatant. All of the finer colloidal particles coalesced together to form bigger floccules and thus improving its removal either by centrifugation or other filtration techniques. However, along with flocculated impurities, even product was precipitating out or was stuck physically to floccules and was getting lost during centrifugation or filtration.

A strategy was devised to keep the protein in solution and yet not to dissolve the flocculated solids. This was bit challenging as solubilizing protein could have potentially dissolved solids too. However, the fine balance was achieved by optimizing the concentrations of Urea and Triton X-100 to keep the protein in solution without dissolving flocculated solids. This ensured optimal protein recovery and supernatant sample with turbidity at NTU of 20 to 100 just after centrifugation. After such clarification the sample remains stable with respect to turbidity for more than 7-8 days when stored at 2-8° C.

### Definitions

Unless otherwise defined herein; the scientific and technical terms used in connection with the present invention shall have the meanings that are, commonly understood by those of ordinary skill in the art. Further, unless otherwise required by context, singular terms shall include pluralities and plural terms shall include the singular. The methods and techniques of the present invention are generally performed according to conventional methods well known in the art. The nomenclatures used in connection with, and techniques described herein are those commonly used in the art. The methods and techniques of the present invention are generally performed according to conventional methods well known in the art.

The term “Pichia pastoris” refers to a species of methylotrophic yeast which is frequently used as an expression system for the production of proteins.

The term “recombinant protein” refers to a protein with an altered/modified genetic sequence which is cloned and expressed in a suitable host system.

The term “primary recovery” or “primary treatment” refers to a process in the clarification of the fermentation supernatant, wherein the harvested broth is treated with chemicals and/or co-solubilizing agents like Urea, Triton X-100 to undergo flocculation, followed by several pH adjustment and centrifugation steps to remove the floccules.

The term “downstream purification” refers to the recovery and purification of biosynthetic pharmaceutical products from related impurities and wastes incurred during the production

The term “human insulin” refers to a human hormone whose structure and properties are well known. Human insulin has two polypeptide chains that are connected by disulphide bridges between cysteine residues, namely the A-chain and the B-chain. The A-chain is a 21 amino acid peptide and the B-chain is a 30 amino acid peptide, the two chains being connected by three disulphide bridges: one between the cysteines in position 6 and 11 of the A-chain, the second between the cysteine in position 7 of the A-chain and the cysteine in position 7 of the B-chain, and the third between the cysteine in position 20 of the A-chain and the cysteine in position 19 of the B-chain.

The term “analogue” or “derivative” in relation to a parent polypeptide refers to a modified polypeptide wherein one or more amino acid residues of the parent polypeptide have been substituted/deleted/added by other amino acid residues. Such addition, deletion or substitution of amino acid residues can take place at the N-terminal of the polypeptide or at the C-terminal of the polypeptide or within the polypeptide. Examples of insulin analogue are insulin Aspart, insulin Lispro, insulin Glargine, oral insulin tregopil etc. Other examples are porcine or bovine insulin which are both analogues of human insulin.

The term “Glargine” particularly refers to the long-acting human insulin analogue which differs from human insulin in which, the amino acid asparagine at position 21 on the Insulin A-chain is replaced by glycine and two arginine residues are added to the C-terminus of the B chain

The term “Lispro” particularly refers to rapid-acting human insulin analogue which chemically differs from human insulin. In insulin Lispro, the amino acid proline at position B28 is replaced by lysine and the lysine in position B29 is replaced by proline.

The term “CIEX Chromatography” refers to ‘Cation Exchange Liquid chromatography’ which is a chromatography process wherein separation is carried out due to the affinity of the positively charged ions towards a negatively charged resin. The chromatography used to capture and concentrate the protein of performing the Fermentation end supernatant clarification while performing the Primary treatment.

The term “NTU” refers to ‘Nephelometric Turbidity Unit’ which is the measure of cloudiness or haziness of a liquid medium caused by finely suspended colloidal particles. The NTU of a solution is measured using a nephelometer.

The term “Flocculation” refers to a process wherein, fine particles are allowed to clump together to form a floc or flocs, which can be separated by various methods like sedimentation or filtration.

The term “Cold temperature” or “cold hold” refers to a temperature in the range of 2° C. to 8° C. The temperature may for example be from 4° C. to 6° C., including 5° C.

The term “Room temperature” or “RT” or “RT hold” refers to an ambient temperature or a temperature in the range of 22° C. to 25° C., including 23° C. or 24° C.

The term “Depth filtration” refers to a type of filtration technique where a porous filtration medium can retain particles when the fluid to be filtered contains a high load of particulate matter. The filter used throughout the medium and as a result can retain a large mass of particles before becoming clogged.

The term “DE Diatomaceous Earth (DE) filtration” refers to a process that uses diatoms or diatomaceous earth—the skeletal remains of small, single-celled organisms—as the filter media.

The term “centrifugation” refers to the technique which involves the application of centrifugal force to separate particles from a solution according to their size, shape, density, viscosity of the medium and rotor speed.

The term “v/v” refers to Volume/Volume. It indicates that the solute and the solvent are liquid in nature. The % v/v, that means the solvent is 100 mL. A percent v/v solution is calculated by the following formula using the milliliter as the base measure of volume (v):

$$\% v / v = mL of solute / 100 mL of solution$$

For clarifying a fermentation supernatant, initially a physical separation method such as membrane filtration has been used. However, this filtration technique had challenges due to the varying nature of the particles (like suspended particles, cells, cell debris, fine colloidal solids, protein aggregates and other insoluble matters) present in the fermentation supernatant post cell separation step. The filtration area requirement for handling this crude supernatant was below 100 L/m² and the quality of feed post filtration was not suitable for a direct load on capture chromatography. The filtration scheme used generally involved usage of 4-5 u filters/1.2 u filters/0.45 u (nominal) membrane filters connected in series. As a result, it has to be concluded that the use of membrane filters is a rather nonviable and uneconomical way of clarification of fermentation supernatants. Similar challenges were experienced with depth filters. Due to the broad distribution of particle size, frequent and abrupt depth filter choking was commonly observed, resulting in extremely low filtrate output per unit filter area.

The challenges that were faced during membrane filtration or depth filtration was mainly attributed to the fact that the supernatant had particulate matter with a broad particle size distribution. Due to this broadness of the distribution, finer particles would choke the filter membrane at the start of filtration and thus decrease the filter throughput tremendously.

Microfiltration is another widely used technique for clarification of feeds with high solid content. Extensive development was done to develop clarification process using microfiltration. A process was developed using 0.1-micron Micro-filtration membrane. Post microfiltration, filtrate was further concentrated through ultrafiltration to overcome the dilution encountered during microfiltration. This process was scaled-up with a total area of 100 m² to filter supernatant volume of 20-22KL. The filtration of the supernatant was achieved in 80-100 hrs with significantly improved clarity. However, the clarified supernatant post microfiltration showed limited stability. Upon storage of micro-filtered supernatant at either room temperature or cold conditions, micro precipitates would reappear changing the sample clarity and turbidity and thus causing clogging of the subsequent capture column.

In summary, conventional centrifugation and/or filtration approaches that were followed to clarify the supernatant, posed the following challenges before applying it to chromatography:
Higher residual solids in the feed caused higher backpressure on capture column;
NTU or suspended colloidal particles kept increasing during the sample hold;

Process time and column life were severely impacted resulting in variable process. As mentioned in Europe patent no. 3070472, certain ionic polymers, specifically cationic polymers, can be used for the flocculation of cell and/or cell debris, as well as for the precipitation/coagulation of proteins. Ionic polymers have also been used to modify fermentation media to enhance the removal of impurities from process streams in applications such as depth filtration or membrane absorber. However, it is also known that as the fermentation media is processed, the pH and conductivity of the media keeps changing. As a result, the effectiveness of these flocculants is typically reduced.

As mentioned in Europe patent no. 1934242, conventional biopharmaceutical protein purification methods used to remove cells and cellular debris are not always effective and sometimes significantly bind the product of interest, increasing the overall process time which can be challenging during the scaling up of the operational procedure. Any improvements that allow for shorter recovery times and/or greater recovery is advantageous, as it reduces costs associated with protein manufacturing.

Another major approach that is commonly applied is flocculation using pH, anionic and cationic agents. In all such flocculation methods, a major problem generally encountered is the co-precipitation of product along with impurities. In such situations, when flocculated impurities were separated physically by means of either centrifugation or filtration, precipitated product was also removed along with impurity precipitate resulting in huge product losses. This was majorly attributed to the phenomenon of physical adsorption or nonspecific interaction of protein of interest to floccules or actual product precipitation. This posed a severe challenge to the flocculation-based clarification approach due to its impact on process cost by causing loss of protein of interest.

In a process disclosed herein a fermentation broth is used. The fermentation broth may be a supernatant obtained by centrifugation of recombinant host cells that produced a recombinant protein. In some embodiments the process may include producing the recombinant protein using the host cells.

Generally, any desired recombinant protein may be included in the fermentation broth. In some embodiments the recombinant protein is insulin or an analogue/derivative thereof. The respective protein may have been expressed in any suitable host cell, such as a eukaryotic system An example of a suitable eukaryotic host cell is a yeast, such as Pichia pastoris.

The process includes adding urea and a non-ionic surfactant to the fermentation broth.

A non-ionic surfactant is a compound that does not have an ionic functional group. Accordingly, its hydrophilic head group is uncharged. Any non-ionic surfactant can generally be used. It may for example be an ether and/or include hydroxyl groups. In some embodiments a non-ionic surfactant is a polyether. In some embodiments a non-ionic surfactant is an amine oxide or a phosphine oxide. In some embodiments a non-ionic surfactant is a sulfoxide.

In some embodiments the non-ionic detergent interchangeably referred as non-ionic surfactant is commercially available under the name Triton, Tween or Brij, such as Brij 35, C12E23, or Polyoxyethylene (23) lauryl ether. Non-ionic surfactants on a polyoxyethylene basis are for example available under the trade name Brij 35, Brij 58, Triton X-100, IGEPAL CA-630 (formerly Nonidet P-40). In an illustrative embodiment the non-ionic detergent is available under the name Triton X-100.

Non-ionic surfactants carrying a plurality of hydroxy groups are for instance ([N,N′-Bis(3-D-gluconamidopropyl)deoxycholamide]), available under the trade name Deoxy Big CHAP, or N,N-bis-(3-D-Gluconamidopropyl)cholamide available under the trade name Big CHAP.

Further examples of non-ionic surfactants carrying a plurality of hydroxy groups are Acyl-N-methylglucamide (MEGA) compounds, such as N-decanoyl-N-methylglucamine or N-octanoyl-N-methylglucamide.

A further suitable non-ionic surfactant is dimethyldidecylphosphine oxide, available under the trade name APO-12. Octyl beta glucoside is another example of a non-ionic surfactant. Another suitable non-ionic surfactant is n-Dodecanoylsucrose. Two further suitable non-ionic surfactants are n-Dodecyl-β-D-glucopyranoside and n-Dodecyl-β-D-maltoside. Another two suitable non-ionic surfactants are Cyclohexyl-n-ethyl-β-D-maltoside and Cyclohexyl-n-hexyl-β-D-maltoside. Cyclohexyl-n-methyl-β-D-maltoside and n-Decanoylsucrose are two further examples of a suitable non-ionic surfactant. Yet another suitable non-ionic surfactant is Digitonin.

Generally, the non-ionic surfactant is added to a final concentration in the range from above 0 to 0.5% v/v. In some embodiments the final concentration of the non-ionic surfactant is above 0.05% v/v, such as above 0.1% v/v. In some embodiments the final concentration of the non-ionic surfactant is up to 0.6% v/v, including up to 0.4% v/v. As an illustrative example, the final concentration of the non-ionic surfactant may be 0.25% v/v.

Urea may be added to a final concentration in the range from above 0 to 0.5 M. In some embodiments the final concentration of urea may be in the range from 0.1 to 0.3M. In some embodiments the final concentration of urea is above 0.02 M, such as above 0.05 M. In some embodiments the final concentration of urea is above 0.1 M, such as above 0.12 M. In some embodiments the final concentration of the non-ionic surfactant is up to 0.35 M, including up to 0.2 M. In some embodiments the final concentration of urea may be in the range from 0.15 to 0.25 M. As an illustrative example, the final concentration of urea may be 0.1 M.

The process further includes adjusting the pH of the fermentation broth, to which urea and the non-ionic surfactant have been added. The pH may for example be adjusted to a pH value in the range from pH 2.5 to pH 4.0. The pH may also be adjusted to a pH value in the range from pH 3.0 to pH 3.8. The pH may for instance be adjusted to a pH of 2.8 or 3.5. In some embodiments the pH may be adjusted to a pH value in the range from pH 7.8 to pH 8.2. The pH may also be adjusted to a pH value in the range from pH 8.0 to pH 8.5.

Any acid or base may be used to adjust the pH value of the fermentation broth. Where the pH needs to be raised, an organic or inorganic base may be added to the fermentation broth. Two suitable bases for pH adjustment are for example sodium hydroxide and potassium hydroxide. The concentration of sodium hydroxide may be in the range from 1 to 4 M, such as 2.5M.

After pH adjustment, the fermentation broth, to which urea and the non-ionic surfactant have been added, is incubated for a time sufficient to allow flocculation to occur. In some embodiments the fermentation broth is incubated for 30 minutes or more, including 1 hour or more. In some embodiments the fermentation broth is incubated for 2 hours or more, including 4 hours or more.

Following incubation, the fermentation broth is separated into soluble and insoluble matter. Thereby insoluble matter is being removed from the fermentation broth, and a solution is obtained that is for ease of reference in the following addressed as a supernatant. Separation into soluble and insoluble matter is generally achieved using physical means. The fermentation broth may be centrifuged (Instrument: Beckman coulter) at a 8983 g force that allows sufficient removal of the flocculation. Following incubation, the fermentation broth may also be exposed to filtration (3M™ Zeta Plus™ capsule, 60SP Nominal Pore Size rating of 0.3 Micron to 4 Micron). The filter may for example be a membrane that allows sufficient removal of the flocculation.

After the pH-treated fermentation broth has been exposed to enhanced gravitational force by centrifugation and filtration or centrifugation, the process may be complete. If desired, or in case flocculation is still being observed, a second adjustment of the pH of the supernatant may be performed.

The pH of any second or further subsequent pH adjustment is selected independently from the pH used for adjusting the pH of the fermentation broth, to which urea and the non-ionic surfactant had been added. As an illustration, where the pH had been increased in the first pH adjustment, the pH may be either increased or decreased in a second or further subsequent pH adjustment. In some embodiments the first pH adjustment may for example be to a pH value in the range from 2.0 to 4.5, and a subsequent pH adjustment may be to a pH value in the range from 7.5 to 8.5. In some embodiments the first pH adjustment may for example be to a pH value in the range from 7.5 to 8.5, and a subsequent pH adjustment may be to a pH value in the range from 2 to 4.5. In some embodiments both the first pH adjustment and a subsequent pH adjustment may be to a pH value in the range from 7.5 to 8.5, but to different pH values within this range. Likewise, the first pH adjustment and a subsequent pH adjustment may be to a pH value in the range from 2.0 to 4.5, but to different pH values within this range.

Generally, in any second or further subsequent pH adjustment, the pH may for example be adjusted to a pH value in the range from pH 7.8 to pH 8.2. The pH may also be adjusted to a pH value in the range from pH 8.0 to pH 8.5. In some embodiments the pH may be adjusted to a pH value in the range from pH 2.5 to pH 4.0. The pH may also be adjusted to a pH value in the range from pH 3.0 to pH 3.8. The pH may for instance be adjusted to a pH of 2.8 or 3.5.

After a second or further subsequent pH adjustment, the supernatant is incubated for a time sufficient to allow further flocculation to occur. In some embodiments the supernatant is incubated for 30 minutes or more, including 1 hour or more. In some embodiments the supernatant is incubated for 2 hours or more, including 4 hours or more.

After a second or further subsequent pH adjustment, the supernatant is again incubated for a time sufficient to allow flocculation to occur. This second or further incubation may last for 30 minutes or more, including 1 hour or more. In some embodiments the second or further incubation may last for 2 hours or more, including 4 hours or more.

Following a second or further incubation, the supernatant may be centrifuged at a g force that allows sufficient removal of the flocculation. A similar g force as detailed above may be used. Following incubation, the supernatant may also be exposed to filtration. A filter as detailed above may be employed. The filter may for example be a membrane that allows sufficient removal of the flocculation.

In some embodiments there is provided a yeast cell culture harvest method, comprising culturing Pichia pastoris cells expressing a recombinant protein in a cell culture medium for a predetermined time or until a desired cell density and/or packed cell volume is achieved, removing cells through centrifugation to obtain cell free supernatant adding urea and a non-ionic surfactant, such as Triton X-100, to the cell free fermentation supernatant and initiating pH based flocculation, mixing the cell free supernatant during flocculation, allowing the flocculent to settle, and recovering the clarified supernatant.

Subsequently the recombinant protein may be exposed to further downstream processing steps, which will typically include chromatography.

### EXAMPLES

In the following, an embodiment of the process disclosed herein is described by way of an example. The protein of interest, such as insulin or an analogue/derivative thereof, was expressed in a yeast expression system, with the yeast selected being Pichia pastoris. The expression of the protein was carried out in fermentation reactors with capacity in the range 20-22KL for large scale production. The protein was secreted out of the cell into the media, in the form of precursors.

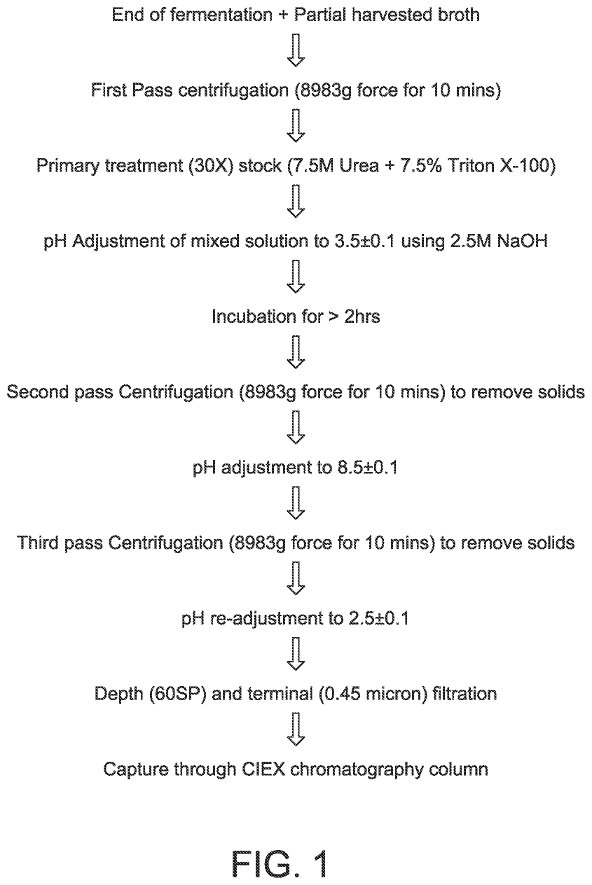

At the end of fermentation, the broth was harvested and centrifuged at 8983 g for 10 mins. The supernatant collected post centrifugation still contained soluble as well as insoluble substances along with the protein of interest. Adding flocculation initiating substances at this step could cause the protein to flocculate along with the other media components. Hence, in order to further reduce the process time, an improved process was designed, wherein the supernatant was treated with a solution of Urea and a non-ionic detergent, followed by pH adjustment in the ranges of 2 to 4.5 or of 7.5 to 8.5 using a suitable base, incubation for a stipulated time, and centrifugation to remove the floccules as shown in FIG. 1 and FIG. 3. Based on the clarity of the broth harvested, additional pH adjustment and centrifugation steps may be carried out.

Addition of urea and a non-ionic detergent solution added at a particular pH initiates flocculation, wherein media components, cells, cell debris, colloids and other materials cluster together to form flocs/aggregates of various sizes. This also prevented the protein to be harvested from combining with the flocs.

The base for pH adjustment was sodium hydroxide or potassium hydroxide, mostly sodium hydroxide. The concentration of sodium hydroxide is indicated below.

### Materials and Methods

The table elaborates the materials and the grade of the material used for the experiments performed below.

The table elaborates the reagents and the method of their preparation used for the experiments performed.

TABLE NO. 1
Reagents and their method of preparation

| Sr. No. | Reagent | Method of Preparation |
|---|---|---|
| 1 | 2.5 M Sodium | Measure 850 mL of water and add 100gm of Sodium |
| | Hydroxide (NaOH) | hydroxide pellets. Make up the total volume to 1 L by |
| | solution | adding purified water. |
| 2 | 0.25 M Urea | Measure 900 mL of water and add 15 gm of Urea powder. |
| | | Make up the total volume to 1 L by adding purified water. |
| 3 | 0.1 M Urea | Measure 900 mL of water and add 6 gm of Urea powder. |
| | | Make up the total volume to 1 L by adding purified water |
| 4 | 0.25% Triton X-100 | Measure 990 mL of purified water and add 2.5 mL of |
| | | Triton X-100. Make up the total volume to 1 L by adding |
| | | purified water. |
| 5 | 0.15% Triton X-100 | Measure 990 mL of water and add 1.5 mL of Triton X-100. |
| | | Make up the total volume to 1 L by adding purified water |
| 6 | Primary treatment | The 30 X primary treatment stock contains 7.5 M Urea and |
| | stock (30 X) for | 7.5% Triton X-100 Measure 400 mL of water and add 450 |
| | Insulin Glargine | gm of Urea & 75 ml of Triton X-100. Make up the volume |
| | | to 1 L by adding remaining purified water. |
| 7 | Primary treatment | The primary treatment agent contains 0.1 M Urea and |
| | stock for Insulin | 0.15% Triton X-100 |
| | Lispro | |

The method of centrifugation was repeated for 2 to 3 times in the process for efficient separation of flocculated particles from the solution.
A wide range of pH was scanned to study the solubility pattern of protein.
An effective pH of either pH 2 to 4.5 or pH 7.5 to 8.5 was identified as suitable for selectively precipitating the impurities and colloidal particles.
An optimized individual concentration of Urea and Triton X-100 and a mixture of Urea and Triton X-100 was chosen as a co-solubilizing agent.
Using Urea and Triton X-100, selective flocculation of impurities and other colloidal particles were triggered, keeping the main product in the solution with minimal loss.

### Process of Flocculation for Fermentation Broth Containing Insulin Glargine

FIG. 1 shows the step by step flow chart of the process of flocculation for fermentation broth containing Insulin Glargine. The Insulin Glargine supernatant from the fermentation broth was further clarified using primary treatment stock (30× stock of Urea and Triton X-100 (as shown in Table 1) at different strengths & at different pH.

Upon the end of fermentation, the fermentation broth was harvested and centrifuged first. Upon centrifugation, mixture of Urea and Trion X-100 from 30× stock was added to the cell free fermentation supernatant. The quantity of reagents is added on volume/volume basis. The mixture was kept for incubation for 2 hours after pH adjustment to 3.5±0.1 by using 2.5M sodium hydroxide solution. The pH depends on the insulin or insulin analogue present in the fermentation broth.

Post incubation the mixture was centrifuged to remove solids in the form of floccules formed at pH 3.5. After this centrifugation, the pH of the supernatant obtained was further adjusted to 8.5±0.1 by using 2.5M sodium hydroxide solution. The second pH adjustment was done to remove additional solids from solution that uniquely flocculate only in the pH range of 7.5 to 8.5. This way, sample goes through two types of flocculation processes each followed by a centrifugation step. This approach of two step flocculation was followed for Insulin Glargine, whereas for most of the other analogues single flocculation step is sufficient to pull-out solids from the cell free supernatant.

The pH of the flocculated supernatant obtained after the last centrifugation step was readjusted to pH of 2.5±0.1.

The mixture was further clarified using depth and terminal filtration followed by cation exchange liquid chromatography.

The method provided herein was further elaborated with the help of experiments. However, these experiments should not be construed to limit the scope of present invention.

In the following experiments, efficiency of flocculation is expressed in terms of percentage of product recovery at primary recovery steps and NTU.

### Experiment 1—Primary Treatment of Glargine Supernatant for Better Clarification & Optimal Recovery

In this experiment, primary treatment of Glargine was carried out at different pH values and stock concentrations. The primary treatment was also carried out in two different batches. Turbidity of the solution was measured using a Nephelometer and the resulting turbidity was reported in Nephelometric Turbidity Units (NTU). The process recovery was calculated for each value from both the batches to evaluate the optimized pH value.

At the end of fermentation, the broth was harvested and centrifuged at 8983 g for 10 mins (to remove the cells and cell debris from the broth), followed by the addition of a solution of Urea and Triton-X-100 to the supernatant from the 30× stock. After this addition, the pH was adjusted to 3.5±0.1 using 2.5M Sodium Hydroxide solution. Once the pH was adjusted, the broth was incubated for 2 hrs, following which the broth was centrifuged at 8983 g for 10 mins. Post completion of centrifugation after pH 3.5 incubation, pH was increased to 8.5 using 2.5 M Sodium Hydroxide solution and was incubated for additional 2 hours and allowed to flocculate. These flocculates formed after pH 8.5 incubation were removed by one more round of centrifugation. This process was followed in trial no 5 referred in below table. All other trials were incubated at a single pH with or without primary treatment stock. This was followed by pH re-adjustment to pH 2.5±0.1 as shown in FIG. 1. Ten trials were conducted in two batches. Primary treatment stock was not added to trials 1, 2 and 3.

Table 2 elaborates the results observed after the performing the trials as per experiment 1.

TABLE 2
Result of the trials for Primary treatment of Glargine supernatant

| Trial details | | | Primary Treatment Attributes | | | |
|---|---|---|---|---|---|---|
| | | Primary | Batch 1 | | Batch 2 | |
| | | treatment | % Primary | | % Primary | |
| Trial no. | pH | stock | recovery | NTU | recovery | NTU |
| 1 | 2.5 | 0X | 95.68% | 183 | 95.55% | 124 |
| 2 | 3.5 | 0X | 95.08% | 166 | 93.33% | 123 |
| 3 | 4.5 | 0X | 77.13% | 124 | 55.78% | 114 |
| 4 | 2.5 | 1X | 93.68% | 65.7 | 91.94% | 48.3 |
| 5 | 3.5 | 1X | 94.19% | 33.9 | 91.16% | 32.3 |
| | followed | | | | | |
| | by 8.5 | | | | | |
| 6 | 3.5 | 1X | 93.95% | 52.2 | 97.63% | 44.4 |
| 7 | 4.5 | 1X | 91.88% | 36.0 | 86.94% | 32.4 |
| 8 | 2.5 | 2X | 94.90% | 35.8 | 92.90% | 33.4 |
| 9 | 3.5 | 2X | 94.90% | 37.5 | 91.83% | 35.9 |
| 10 | 4.5 | 2X | 92.67% | 35.1 | 97.02% | 30.0 |

As seen in Table 2, there was a remark-able difference in the primary recovery at pH 4.5, with (Trial no. 5, 7 and 10) and without co-solubilizing agents (Trial no. 3 & 7). It was observed that the addition of co-solubilizing agents (primary treatment stock) during the primary treatment process, product loss can be minimized to a large extent. It was also observed that pH 3.5- and 8.5 and pH 3.5 (standalone) were found to be the most optimized process conditions for Primary clarification, as these trials (Trial no. 5 & 6) offer a fine balance between the NTU and process recovery.

### Experiment 2—Stability of Glargine Clarified Supernatant Via Primary Treatment Approach (NTU Stability)

In order to determine the stability of the clarified supernatant after primary treatment, separate set of experiments were carried out with respect to NTU. The experiments were carried out in three trials, with different pH values and different urea and Triton-X concentrations. The experiments were performed at two different hold conditions such as cold temperature i.e. 5±3° C. and room temperature i.e. 22±3° C.

Table 3 elaborates the steps performed in the trials

TABLE NO. 3
Process details

| | | |
|---|---|---|
| Trial 1 | Trial 2 | Trial 3 |

1^{st} Pass supernatant

| | | |
|---|---|---|
| pH was adjusted | pH was adjusted | pH was adjusted |
| to 3.5 | to 3.5 | to 2.5 |
| ↓ | ↓ | ↓ |
| Addition of Urea (0.25 M) | Without addition of | Without addition |
| and Triton-X-100 | Urea and Triton- | of Urea and |
| (0.25%)-1X (✓) | X-100-0X (x) | Triton-X-100-0X (x) |

Processed for 2^{nd} pass Centrifugation (8983 g & 10 minutes) &
pH was adjusted to 2.5 ± 0.2
NTU stability was performed at cold temperature and RT.

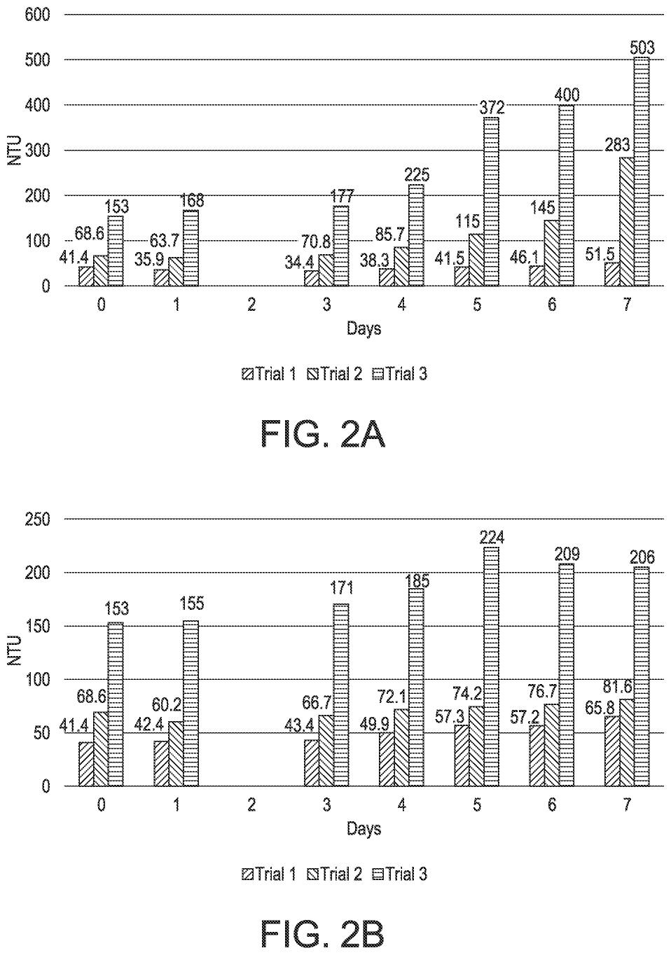

Table 4 elaborate the details of trials conducted for the process elaborated in table 3. The results are better illustrated when observed along with FIG. 2A and FIG. 2B.

TABLE 4
NTU stability data on hold (RT& cold)

| | Trial 1 | | | Trial 2 | | | Trial 3 | |
|---|---|---|---|---|---|---|---|---|
| | Cold | RT | Day | Cold | RT | Day | Cold | RT |
| Day | NTU values | | | | | | | |
| 0 | 41.4 | 41.4 | 0 | 68.6 | 68.6 | 0 | 153 | 153 |
| 1 | 42.4 | 35.9 | 1 | 60.2 | 63.7 | 1 | 155 | 168 |
| 2 | | | 2 | | | 2 | | |
| 3 | 43.4 | 34.4 | 3 | 66.7 | 70.8 | 3 | 171 | 177 |
| 4 | 49.9 | 38.3 | 4 | 72.1 | 85.7 | 4 | 185 | 225 |
| 5 | 57.3 | 41.5 | 5 | 74.2 | 115 | 5 | 224 | 372 |
| 6 | 57.2 | 46.1 | 6 | 76.7 | 145 | 6 | 209 | 400 |
| 7 | 65.8 | 51.5 | 7 | 81.6 | 283 | 7 | 206 | 503 |

Trial 1 was subjected to primary treatment post pH adjustment to 3.5 as elaborated in table 4.

The pH was adjusted to 3.5 and 2.5 in trial 2 and trial 3 respectively without primary treatment agents.

All the trials were processed for 2^{nd} pass centrifugation at 8983 g for 10 mins following which the pH was adjusted to 2.5±0.2

As per the performance of the trials, the NTU was stable for seven days.

As evident from the above stability data (Table 4). NTU was found to be almost stable for 7 days (with minor increase) at cold hold for trial 1 (pH 3.5 with Primary treatment approach), and trial 2 (pH 3.5 without Primary treatment approach). At the same time point it was found to be increasing for trial 3 (pH 2.5 without Primary treatment approach)

During RT hold, Trial 1 of pH 3.5 with primary treatment approach showed better stability as compared to other trials (pH 3.5 without primary treatment approach & pH 2.5 without Primary treatment approach), which clearly indicated that primary treatment offers a great advantage with respect to NTU stability.

### Experiment 3: Depth Filtration Trial for Glargine Clarified Supernatant—Comparative Evaluation

Higher turbidity of the supernatant in terms of NTU can pose a greater challenge during the depth filtration stage, thereby requiring larger filter area and considerably the higher process time. Filtration throughput was studied for the different (with & without primary treatment) trials of experiments as given below in table 5.

As elaborated in the Table 5, the trials were put through depth filtration (3M™ Zeta Plus™ capsule, 60SPNominal pore size rating of 0.3 Micron to 4 Micron) operation and filtration throughput data was observed at a cut off pressure limit of 2.0 bar. Trial 1 depth filtration data was generated in lab, while remaining 2 data seta sets (pH 3.5-8.5 & pH 3.5, both with Primary treatment) were referred from at scale batches (past manufacturing runs).

TABLE 5
Filtration throughput data

| Primary Recovery | | | | |
|---|---|---|---|---|
| process conditions | | | | |
| | | Primary | Observed Throughput | |
| | | treatment | at 2 bar pressure limit | |
| Trial No. | pH | stock | Batch 1 | Batch 2 |
| 1 | 2.5 | 0 X | 252 L/m2 | 176 L/m2 |

| | | | |
|---|---|---|---|
| 2 | 3.5 | 1 X | 4459 L/m2 |
| | followed by | | |
| | 8.5 | | |
| 3 | 3.5 | 1 X | 1407 L/m2 |

Result—Trial 1 with pH 2.5 without any Primary treatment, showed the least filtration throughput as compared to other arms as shown in above table. The volumetric throughput of the trial 1 (clarification approach without any primary treatment at pH 2.5) was found to be 252 L/m² & 176 L/m2 respectively for Batch 1 & 2, while other arms with primary treatment approach (pH 3.5 & 3.5 followed by 8.5) yielded higher volumetric throughput (more than 1000 L/m²) in both the cases.

This showed that performing the primary treatment process has four advantages as follows:
1. increases the process recovery;
2. provides a better clarification;
3. increases the filtration throughput by a great extent, and;
4. Reduces the batch running cost.

### Process of Flocculation for Cell Free Fermentation Supernatant Containing Insulin Lispro

FIG. 3 shows the step by step flow chart of the process of flocculation for cell free fermentation supernatant containing Insulin Lispro. The Insulin Lispro cell free fermentation supernatant was obtained by centrifugation of broth at 8983 g for 15-30 min.

The pH of cell free fermentation supernatant obtained after first broth centrifugation was adjusted from 6-6.5 (fermentation pH) to 2.0±0.1 by using Ortho phosphoric Acid/2.5 M Sodium hydroxide solution. After pH adjustment, this supernatant mixture was left for 8-12 hrs of incubation (static hold) for causing a flocculation of certain type of fermentation impurities or media components or salts. Uniquely in this first stage of flocculation performed at pH 2, there is no need for addition of Primary Treatment agents (Urea/Triton-X-100) as the product is highly soluble at this pH condition and thus doesn't co-precipitate with impurities or salts. After incubation for almost 12 hours, flocculated solids are removed by Centrifugation. The supernatant at pH 2 obtained after centrifugation is further adjusted to various pH conditions to explore second step of flocculation i.e. at pH 3.5, 4 and 4.5. Before pH adjustment, sample is added with Primary treatment agents on basis to avoid product precipitation. This sample at pH 3.5, pH 4 or at pH 4.5 with primary treatment agents is incubated for 2-4 hours for flocculation to take place and then centrifuged for 15-30 min at 8983 g. Sample obtained after last centrifugation was readjusted to pH 2.5 for further filtration through depth and terminal filters and subsequently loading on to Cation exchange chromatography column for capture.

### Experiment 4: Primary Treatment of Lispro Fermentation Supernatant for Better Clarification & Optimal Recovery

Lispro fermentation supernatant was clarified using Primary treatment stock (30× stock of Urea and Triton-X-100 i.e. 3M Urea and 4.5% Triton-X-100)) at different strengths & at different pH as per the process flow described in FIG. 3. Table 6 shows the outcome of these trials.

TABLE 6
Primary treatment observation for Lispro

| Trial Details | | | Primary Treatment Attributes | |
|---|---|---|---|---|
| | | Primary | Primary | |
| | | treatment | recovery | |
| Trial No. | pH | stock | (Yield %) | NTU |
| 1 | 2.0 | 0 X | 76.26% | 103 |
| 2 | 4.0 | 0 X | 80.11% | 106 |
| 3 | 2.0 | 1 X | 76.90% | 7.3 |
| 4 | 3.5 | 1 X | 87.18% | 100 |
| 5 | 4.0 | 1 X | 83.94% | 104 |
| 6 | 4.5 | 1 X | 83.51% | 97.3 |

Result: Considering the NTU and process recovery as the two main evaluation criteria, clarification trials (3 & 6) with primary treatment approach at pH values 2.0 and 4.5 respectively (with IX strength of Urea and Triton-X-100), showed better process recovery and clarification compared to other combinations.

### Experiment 5: Stability of Lispro Clarified Supernatant Via Primary Treatment Approach (NTU Based Stability Monitoring)

Separate set of experiments were performed to determine the stability of clarified supernatant with respect to NTU. Stability of the clarified Lispro supernatant was performed with respect to the NTU at two different hold conditions, i.e. at cold temperature (5±3° C.) and RT (24±2° C.).

The experiments were performed in sets of two trials viz.
Trial 1 and Trial 3 with Primary treatment carried out/performed at pH 2.0±0.2 and pH 4.0±0.2 respectively.
Trial 2 and Trial 4 without Primary treatment carried out/performed at pH 2.0±0.2 and pH 4.0±0.2 respectively.

Trial 1 and 3 were carried out with primary treatment, whereas Trial 2 and 4 were carried out without primary treatment.

The steps of process for performing these experiments are elaborated in Table 7 and 8.

TABLE 7
NTU stability of Lispro clarified supernatant at pH 2.0 ± 0.2

| Trial 1-Primary | Trial 2-without Primary |
|---|---|
| treatment at pH 2.0 ± 0.2 | treatment at pH 2.0 ± 0.2 |
| 1^{st} Pass supernatant pH was adjusted to 2.0 ± 0.2 | |
| Processed for 2^{nd} pass Centrifugation (8983 g at for 10 minutes) | |
| Addition of Urea (0.15 M) and | Without addition of Urea and |
| Triton-X-100 (0.15%)-1X | Triton-X-100-0X |

pH was adjusted to 2.0 ± 0.2
3^{rd} pass Centrifugation (6000 g for 10 minutes) was performed &
pH was re-adjusted to 2.5 ± 0.2
NTU stability was performed at cold (5 ± 3º C.) and RT (22 ± 3° C.)

TABLE 8
NTU stability of Lispro clarified supernatant at pH 4.0 ± 0.2

| Trial 3-Primary | Trial 4-Without Primary |
|---|---|
| treatment at pH 4.0 ± 0.2 | treatment at pH 4.0 ± 0.2 |
| 1^{st} Pass supernatant pH was adjusted to 2.0 ± 0.2 | |
| Processed for 2^{nd} pass Centrifugation (6000 g at for 10 minutes) | |
| Addition of Urea (0.15 M) | Without addition of Urea |
| and Triton-X-100 (0.15%)-1X | and Triton-X-100-0X |

pH was adjusted to 4.0 ± 0.2
3^{rd} pass Centrifugation (6000 g at for 10 minutes) was
performed & pH was adjusted to 2.5 ± 0.1
NTU stability performed at cold (5 ± 3º C.) and RT (22 ± 3° C.).

NTU stability was performed at RT (22±3° C.) as shown in table 9 and at cold temperature (5±3° C.) as shown in table 10.

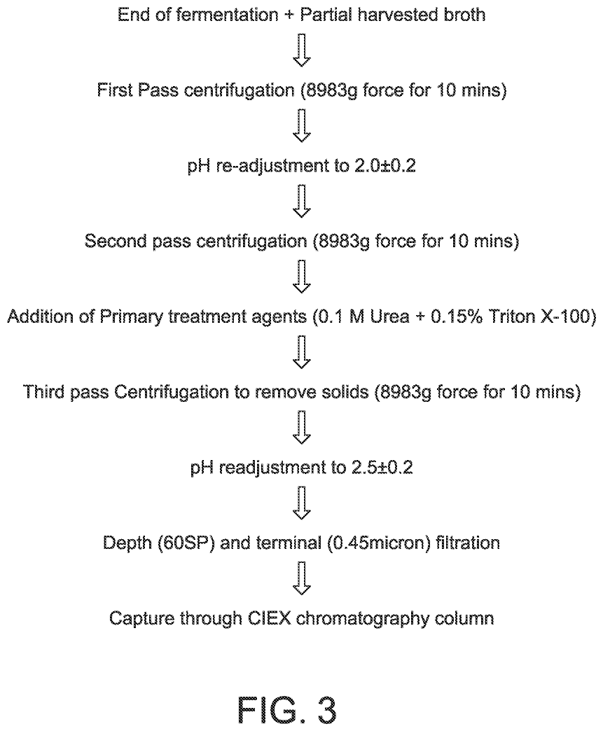

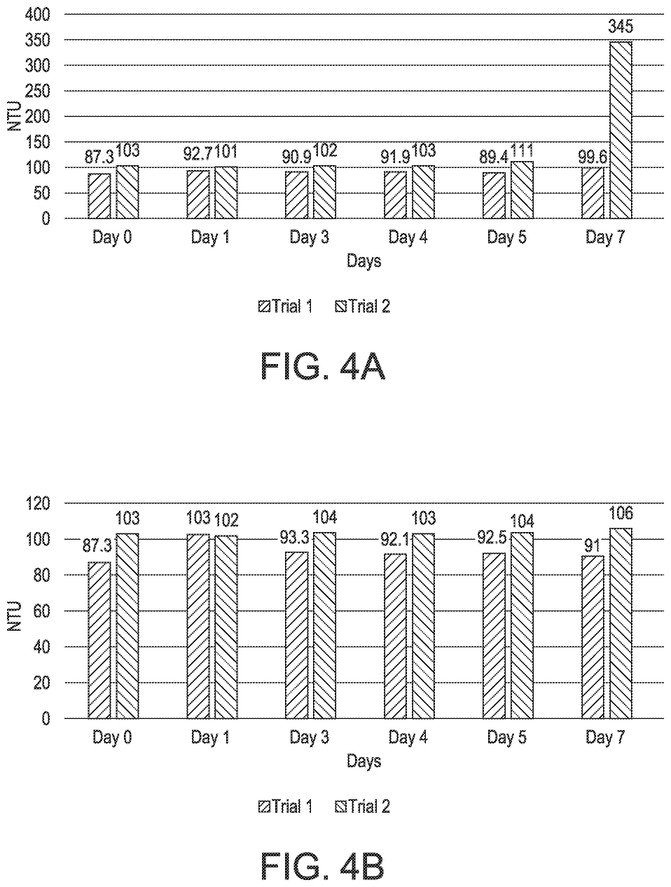

### a) NTU Stability of Clarified Supernatant at RT Hold (22±3° C.)—Results are Illustrated in FIG. 4A.

TABLE 9
NTU stability data at RT hold

| | Trial 1 | Trial 2 |
|---|---|---|
| Day 0 | 87.3 | 103 |
| Day 1 | 92.7 | 101 |
| Day 3 | 90.9 | 102 |
| Day 4 | 91.9 | 103 |
| Day 5 | 89.4 | 111 |
| Day 7 | 99.6 | 345 |

### b) NTU Stability of Clarified Supernatant at Cold Temperature Hold (5±3° C.)—Results are Illustrated in FIG. 4B.

TABLE 10
NTU stability data at cold temperature hold

| | Trial 1 | Trial 2 |
|---|---|---|
| Day 0 | 87.3 | 103 |
| Day 1 | 103.0 | 102 |
| Day 3 | 93.3 | 104 |
| Day 4 | 92.1 | 103 |
| Day 5 | 92.5 | 104 |
| Day 7 | 91.0 | 106 |

Similar set of experiments were carried out at RT with trials 3 and 4. NTU stability was performed at RT (22═3° C.) as shown in table 11 and at cold temperature (5±3° C.) as shown in table 12.

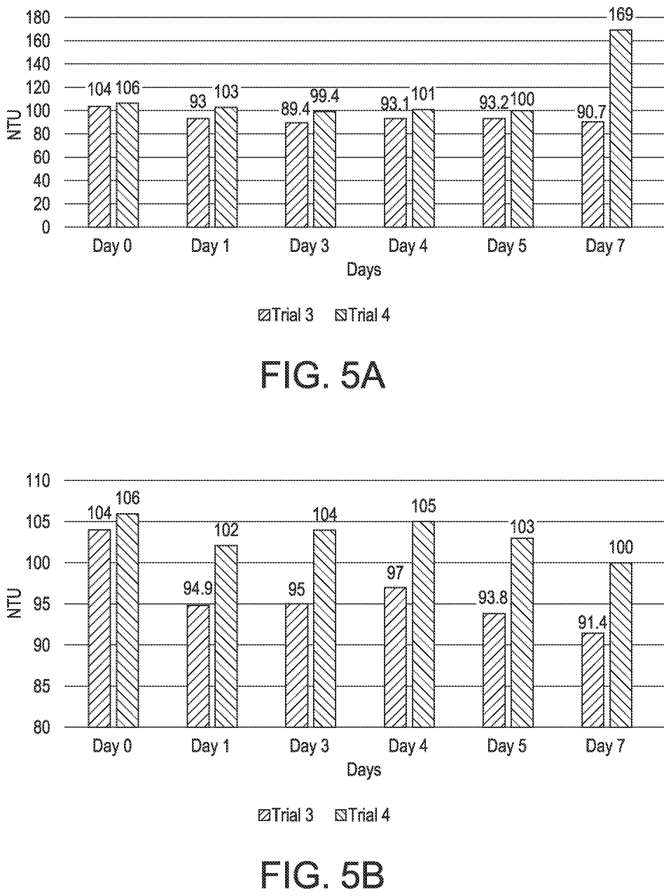

### c) NTU Stability of Clarified Supernatant at Room Temperature Hold (22±3° C.)—Results are Illustrated in FIG. 5A.

TABLE 11
NTU stability data at RT hold

| | Trial 3 | Trial 4 |
|---|---|---|
| Day 0 | 104 | 106 |
| Day 1 | 93.0 | 103 |
| Day 3 | 89.4 | 99.4 |
| Day 4 | 93.1 | 101 |
| Day 5 | 93.2 | 100 |
| Day 7 | 90.7 | 169 |

### d) NTU Stability of Clarified Supernatant at Cold Temperature Hold (5±3° C.)—Results are Illustrated in FIG. 5B.

TABLE 12
NTU stability data at cold temperature hold

| | Trial 3 | Trial 4 |
|---|---|---|
| Day 0 | 104 | 106 |
| Day 1 | 94.9 | 102 |
| Day 3 | 95.0 | 104 |
| Day 4 | 97.0 | 105 |
| Day 5 | 93.8 | 103 |
| Day 7 | 91.4 | 100 |

Result: It was observed that without the addition of Urea and Triton-X-100 stock solution, NTU increases at Room temperature (RT) hold after 5^{th} day onwards. This observation was found to be similar in both the trials without primary treatment i.e. at pH 2.0±0.2 and pH 4.0±0.2

No discernible difference in NTU was found during cold hold for both the arms (with and without primary treatment) at both the pH stages (i.e. pH 2.0±0.2 and pH 4.0±0.2), nevertheless trials with Primary treatment fared better with respect to NTU as compared to the trials without primary treatment.

Conclusion: The process disclosed herein provides a simple approach to flocculation of soluble impurities from a complex fermentation supernatant. It uses simple chemical agents and pH parameters to cause flocculation of soluble impurities. The agents used in flocculation process do not interfere in subsequent chromatographic purification by ion exchange chromatography unlike commercially available flocculants. The disclosed process avoids product loss during flocculation process by use of right mix of urea and detergent thus keeping product of interest in the solution. The flocculation process increases the size of the floccules such that they can be removed by simple physical separation methods like centrifugation. Impurities from solution are removed completely through this method of flocculation which is indicated by stability of clarified solution for long duration of almost five days at room temperature and more than seven days at cold conditions. This novel approach enables negative purification by pulling out impurities from the solution, clarifies it and makes it suitable for chromatographic loading. More than 99% of flocculation agents are removed after capture chromatography, making sure that they do not appear as residuals in final products. Proposed method offers major advantages in reducing process time and cost contribution for primary recovery steps and avoids fouling of capture column. The process of filtration employed before chromatography was positively impacted due to clarification by this primary recovery approach as the filtration capacity of depth filters was increased by 5 to 20 folds. The process of flocculation was scaled up by 1000 folds and performed similar to small scale observations.

1-25. (canceled)

26. A process of purifying a recombinant protein in a fermentation broth, the process comprising:
a) adding urea and a non-ionic detergent to the fermentation broth;
b) adjusting the pH of the fermentation broth to a value in the range from pH 2 to 4.5 or to a value in the range from pH 7.5 to 8.5;
c) incubating the fermentation broth for at least 30 minutes; and
d) separating insoluble matter from the fermentation broth, thereby obtaining a supernatant.

27. The process according to claim 26, further comprising:
a) adding urea and a non-ionic detergent to the supernatant;
b) adjusting the pH of the supernatant to a value in the range from pH 2 to 4.5 or to a value in the range from pH 7.5 to 8.5;
c) incubating the supernatant for at least 30 minutes; and
d) separating insoluble matter from the supernatant.

28. The process according to claim 26, wherein urea is added to a concentration of 0.1-0.3M.

29. The process according to claim 28, wherein urea is added to a concentration of 0.15 to 0.25M.

30. The process according to claim 26, wherein the non-ionic detergent is added to a concentration in the range of 0.1 to 1% (v/v).

31. The process according to claim 26, wherein separating insoluble matter from the fermentation broth and/or the supernatant is performed by centrifugation or filtration.

32. The process according to claim 26, wherein the recombinant protein is insulin or an insulin analogue or derivative.

33. The process according to claim 32, wherein the insulin analogue is Insulin Glargine, Insulin Lispro, Insulin Aspart or oral insulin Tregopil.

34. The process according to claim 26, wherein the recombinant protein has been produced by a yeast, wherein the yeast is Pichia pastoris.

35. The process according to claim 26, wherein the non-ionic detergent is selected from the group of Triton, Tween or Brij series.

36. The process according to claim 35, wherein the non-ionic detergent is Triton-X-100 in a concentration of 0.1-0.4%; wherein the Triton-X-100 is 2-[4-(2,4,4-trimethylpentan-2-yl)phenoxy]ethanol.

37. The process according to claim 36, wherein the concentration of Triton-X-100 is 0.15%.

38. The process according to claim 26, wherein pH adjustment is carried out using a suitable base selected from the group of sodium hydroxide or potassium hydroxide; wherein the concentration of sodium hydroxide is 2.5M.

39. The process according to claim 31, wherein separating insoluble matter from the fermentation broth and/or the supernatant comprises exposing the same to depth filtration.

40. The process according to claim 26, wherein a final capture of the protein is by Cation exchange chromatography.

41. The process according to claim 26, wherein more than 99% of urea and the non-ionic detergent is removed and more than 95% of the final protein is recovered after Cation exchange chromatography.

42. The process of purifying a recombinant protein according to claim 26, wherein the recombinant protein is insulin Glargine, comprising:
a) collecting a fermentation broth obtained after production of insulin glargine using the host Pichia pastoris;
b) adding 0.25M urea and 0.25% (v/v) of Triton X-100 to the fermentation broth;
c) adjusting the pH of the fermentation broth to a value in the range from pH 3.0 to 3.5;
d) incubating the fermentation broth for at least 2 hours;
e) separating insoluble matter from the fermentation broth by centrifugation, thereby obtaining a supernatant;
f) adjusting pH of the supernatant to 8.5;
g) incubating the supernatant for at least 2 hours; and
h) separating insoluble matter from the supernatant by centrifugation.

43. The process according to claim 42, further comprising readjusting the pH to a value of 2.5 and comprising as a final capture of the insulin Glargine purification by cation exchange chromatography.

44. The process of purifying a recombinant protein according to claim 26, wherein the recombinant protein is insulin Lispro, comprising:
a) collecting a fermentation broth obtained after production of insulin Lispro using the host Pichia pastoris;
b) adjusting the pH of the fermentation broth to a value in the range from 2.0 to 2.5;
c) incubating the fermentation broth for at least 30 minutes;
d) separating insoluble matter from the fermentation broth by centrifugation, thereby obtaining a supernatant;
e) adding 0.1 M urea and 0.15% Triton X-100 to the supernatant;
f) adjusting the pH to a value in the range from pH 3.8 to 4.2;
g) incubating the supernatant for at least 30 minutes; and
h) separating insoluble matter from the supernatant by centrifugation;

45. The process according to claim 44, further comprising readjusting the pH to a value of 2.5 and comprising as a final capture of the insulin Lispro purification by cation exchange chromatography.
